(19) United States
(12) Patent Application Publication
(10) Pub. No.: US 2023/0381302 A1
(43) Pub. Date: Nov. 30, 2023

(54) TREATMENT PROTOCOL USING INTRALYMPHATIC IMMUNOTHERAPY

(71) Applicants: Aarhus Universitet, Aarthus C (DK); Region Midtjylland, Viborg (DK); Imperial College Innovations Limited, London (GB)

(72) Inventors: Hans Jürgen Hoffmann, Højbjerg (DK); Søren Helbo Skaarup, Hjortshøj (DK); Johannes Martin Schmid, Risskov (DK); Stephen R. Durham, London (GB)

(21) Appl. No.: 18/031,839

(22) PCT Filed: Aug. 5, 2021
(86) PCT No.: PCT/DK2021/050256

§ 371 Date: Apr. 13, 2023

(30) Foreign Application Priority Data
Oct. 14, 2020 (DK) PA202070691

(51) Int. Cl. A61K 39/36; A61P 37/08

(52) U.S. Cl. CPC A61K 39/36; A61P 37/08; A61K 2039/55505

(57) ABSTRACT

The present invention relates to an improved treatment protocol using Intralymphatic immunotherapy (ILIT). In particular, the present invention relates ILIT in relation to grass allergy.

### TECHNICAL FIELD OF THE INVENTION

The present invention relates to an improved treatment protocol using Intralymphatic immunotherapy (ILIT). In particular, the present invention relates ILIT in relation to grass allergy.

### BACKGROUND OF THE INVENTION

Allergy is a chronic condition affecting more than 20% of westernised society. A common condition is allergic rhino-conjunctivitis that may progress to become asthma if not treated appropriately. About 45% of allergic disease cannot be treated adequately with symptomatic medication like antihistamines and topical steroids. The only solution for these patients is allergen immunotherapy (AIT). Allergic rhinitis of a patient costs society 950€ pa, most in presenteeism.

AIT has been discovered in 1911 and has since then matured to subcutaneous immunotherapy (SCIT), and since then to sublingual immunotherapy. These treatments last three years and are associated with many side effects. SCIT entails many long visits to the doctor. As a result, less than 5% of eligible patients make use of this treatment option.

Intralymphatic Immunotherapy (ILIT) delivers medicine directly to the lymph node, where it changes the immune response to relieve symptoms of the treated patient (reviewed in Senti et al Int Arch Allergy Immunol. 2019; 178(2):141-149, Skaarup S H et al. J Allergy Clin Immunol 2021 March; 147(3):1011-1019. doi: 10.1016/j.jaci.2020.07.002, Skaarup et al Allergy. 2021 June; 76(6):1859-1861. doi: 10.1111/a11.14642 and Hoang M P et al. Intralymphatic immunotherapy for allergic rhinoconjunctivitis: a systematic review and meta-analysis. Rhinology 2021; 59:236-244.). Efficacy of ILIT has been shown in many small trials, most of which use Alutard, a product for subcutaneous immunotherapy marketed by ALK-Abelló Most trials have used 1000 SQ-U with good effect. A single trial has used 3000 SQ-U. The dose of 1000 SQ-U has been selected on the basis of expert opinion.

ILIT has been practised with 1000 SQ-U or higher. The ALK SQ-U unit is clinically defined, but 100 000 SQ-U correspond to 20 μg of the major allergen Phl p 5. 1000 SQ-U thus represent about 0.2 μg major allergen Phl p 5. Attempts by the group of Lars Olaf Cardell at Karolinska Institute and Lund University have explored increasing the dose to 3000, 5000 and 10 000 SQ-U (L Hellkvist, Karolinska Institute, PhD dissertation).

WO 02/28429 A2 describes that the modulation or elimination of an allergic condition can be achieved by injecting small amounts of allergen directly into a lymph node, which greatly reduces potential side effects.

WO 2010/043675 A1 relates to the use of a pharmaceutical product comprising allergen and optionally an adjuvant for fast up-dosing in connection with allergy vaccination wherein a reduced number of injections are used.

Hence, an improved treatment of allergic diseases would be advantageous, and in particular, a more efficient and/or reliable Intralymphatic immunotherapy (ILIT) treatment would be advantageous.

### SUMMARY OF THE INVENTION

Allergic responses, like many other immune responses, have an optimal allergen concentration. Too much is not as good as the optimum amount, and too little is not as good either. This is the case for the IgE mediated basophil and mast cell response that causes pathology in allergy, for T cell responses, B cell responses (which result in allergic response as they produce the IgE antibody that is required) and may well be the case for the dose of allergen that is optimal for AIT where responses that limit the symptoms of allergy are generated.

Allergen is presented by dendritic cells to T cells and to B cells in the lymph node. The concentration of allergen (or any antigen) is determinate for the efficacy of this response. B cells specific for allergen form and may be part of a protective immune response to allergen that the sensitised patient has IgE against in response to this presentation.

However, allergen immunotherapy is a payoff between clinical effect and allergic side effects, even when delivering the allergen directly into the lymph node. Allergic side effects vary over time in the sensitised individual. The reason is the physiologic interface to the immune system and is not yet characterised. For that reason, it is best to reduce the amount of allergen injected into an allergic patient.

The present invention is based on the realization that the current used and tested dosages of allergen in ILIT treatment have focused on increasing the dosage to improve the effect (1000 SQ-U or higher). The present inventing team on the other hand, has realized that an improved effect is feasible with a lower dosage. Such lower dosage could e.g. be combined with a lowered ratio of allergen to adjuvant ratio, to compensate for the lower amount of allergen.

Thus, an object of the present invention relates to the provision of an improved ILIT treatment protocol. Improvement could be in relation to:
Improved vaccination effect (longer and/or stronger); and/or
Better clinical efficacy; and/or
Lower side effects, such as allergic responses.

Thus, one aspect of the invention relates to a pharmaceutical composition comprising 100-400 SQ-U grass allergen, such as grass pollen extract (e.g. containing 0.02 μg Phl p 5 to 0.08 μg Phl p 5), and optionally an adjuvant, for use in the treatment or alleviation of grass allergy, wherein the pharmaceutical composition is administered by Intralymphatic immunotherapy (ILIT) to a subject. Preferably, allergen is provided in the form of an extract, more preferably a grass pollen extract. It can be standardised by the natural content of major allergen Phl p5.

Example 2 shows an improvement in the patient groups receiving a 20 ng Phl p5 dosage or a 60 ng Phl p5 dosage compared to the previous used concentrations (0.2 μg/200 ng Phl p 5) using the standard cSMS scoring system (Pfaar O et al. Allergy 2014; 69:854-867). The best effect was obtained using the 60 ng Phl p5 dosage, reaching a reduction by 67% in cSMS (see also FIGS. 1-3).

The present invention will now be described in more detail in the following.

### BRIEF DESCRIPTION OF THE FIGURES

FIG. 1 shows reduction in cSMS using different concentrations of allergen preparations standardised to the major grass allergen Phl p 5 during ILIT treatment.

FIG. 2 shows a conventional representation of the effect of ILIT at low doses of allergen compared with an open control group.

FIG. 3 shows linear regression of cSMS against log (pollen grains/cubic meter) detected on the day the cSMS was reported with different concentrations of the major grass allergen Phl p 5 during ILIT treatment.

### DETAILED DESCRIPTION OF THE INVENTION

### Definitions

Prior to discussing the present invention in further details, the following terms and conventions will first be defined:

ILIT

Intralymphatic immunotherapy.

Alutard 225

Alutard 225 (Alk-Abello A/S) is an allergen formulation in the form of a suspension containing allergens adsorbed to aluminium hydroxide. The allergens are from Phleum pratense.

Alutard SQ containing 100.000 SQ-U/ml and 1.13 mg Al (aluminium ion)/ml are commercially available. Different types of Alutard compositions are sold by ALK-Abello.

Bio-Potency

In the field of allergy extracts, there is no international accepted standardization method. A number of proprietary units of extract strength i.e. bio-potency exist. The methods employed and the units used normally measure the allergen content and biological activity. Examples hereof are SQ-Units (Standardized Quality units), BAU (Biological Allergen Units), BU (biological units), UM (Units of Mass), IU (International Units) and IR (Index of Reactivity). Hence, if extracts of origins other than those disclosed herein are used, they need to be standardised against extract disclosed herein in order to determine their potency in SQ units or any of the above mentioned units. The subject matter is dealt with in e.g. Larenas-Linnemann et al. Ann Allergy Asthma Immunol. 2008; 100:137-145. An ELISA assay for Phl p5 has recently been proposed to be the European Pharmacopoeia standard method to standardise different grass pollen extracts (Zimmer J et al Allergy. 2021 Jul. 9. doi: 10.1111/all.15003).

The bio-potency, i.e. the in vivo allergenic activity, of a given extract depends on a number of factors, the most important being the content of major allergens in the extract, which varies with the composition of the biological source material. For further information, we refer to e.g. WO 2010/043675.

SQ-Unit (SQU or SQ-U)

The SQ-Unit may be determined in accordance with ALK-Abello A/S's “SQ biopotency”-standardisation method, where 100,000 SQ units equal the standard subcutaneous maintenance dose. Normally 1 mg of extract 5 contains between 100,000 and 1,000,000 SQ-Units, depending on the allergen source from which they originate, and the manufacturing process used. The precise allergen amount can be determined by means of immunoassay i.e. total major allergen content and total allergen activity.

ILIT has been practised with 1000 SQ-U. The ALK SQ-U unit is clinically defined, but 100,000 SQ-U correspond to 20 μg of the major allergen Phl p 5. 1000 SQ-U thus represent about 0.2 μg major allergen Phl p 5 and 100 SQ-U thus represent about 0.02 μg major allergen Phl p 5.

Guidance to the normally applied, acceptable tests measuring bio-potency are found e.g. in Note for Guidance on Allergen Product; The European Agency for the Evaluation of Medicinal Product, CPMP_BWP_243_96, London, 1996.

Allergy Vaccination or Desensitization

As used herein, the terms “allergy vaccination” or “desensitization” include both treatment of existing allergy and prevention of further allergies. By the term “treatment” is meant partly or wholly curing, alleviating symptoms or inhibiting causes of symptoms. The term “prevention” means prophylactic treatment.

cSMS

cSMS is a homogeneous combined symptom and medication score, which is a simple and standardized method that balances both symptoms and the need for antiallergic medication in an equally weighted manner. The scoring system is defined by EAACI, and sanctioned by EMA and FDA (Pfaar 0 et al. Recommendations for the standardization of clinical outcomes used in allergen immunotherapy trials for allergic rhinoconjunctivitis: an EAACI Position Paper. Allergy 2014; 69: 854-867).

It should be noted that embodiments and features described in the context of one of the aspects of the present invention also apply to the other aspects of the invention.

Pharmaceutical Composition

As outlined above, the present invention is based on the realization that the current used and tested dosages of allergen in ILIT treatment has been focusing on increasing the dosage to improve the effect. The present inventing team on the other hand, has realized that an improved effect is feasible with a lower dosage. Such lower dosage could e.g. be combined with a lowered ratio of allergen to adjuvant, to compensate for the lower amount of allergen.

Thus, an aspect of the invention relates to a pharmaceutical composition comprising 100-400 SQ-U grass allergen, e.g. such as grass pollen extract containing/comprising 0.02 μg Phl p 5 to 0.08 μg Phl p 5, and optionally an adjuvant, for use in the treatment or alleviation of grass allergy,
wherein the pharmaceutical composition is administered by Intralymphatic immunotherapy (ILIT) to a subject.

Thus, an aspect also relates to a pharmaceutical composition comprising 0.02-0.08 μg Phl p 5, preferably provided in the form of a grass pollen extract, and optionally an adjuvant, for use in the treatment or alleviation of grass allergy, wherein the pharmaceutical composition is administered by Intralymphatic immunotherapy (ILIT) to a subject.

Thus, it is to be understood that a dosage of 100-400 SQ-U is to be administered to the subject, such as 200-400 SQ-U.

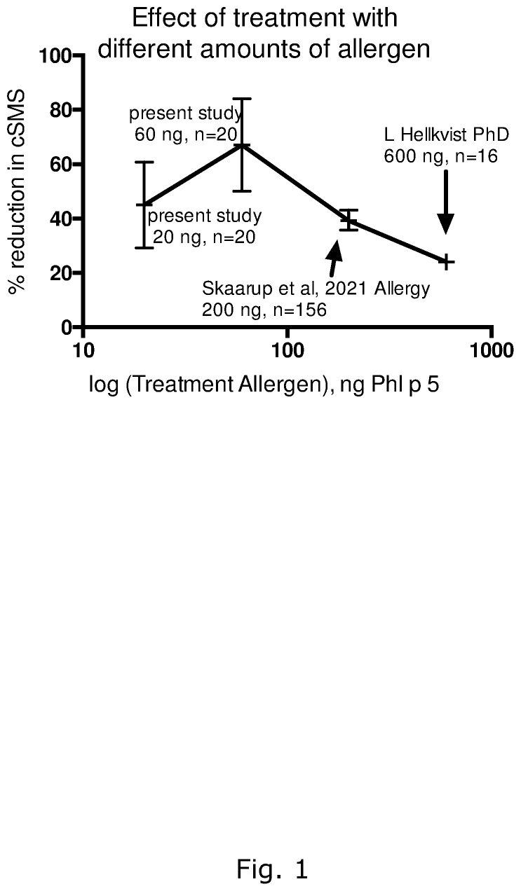

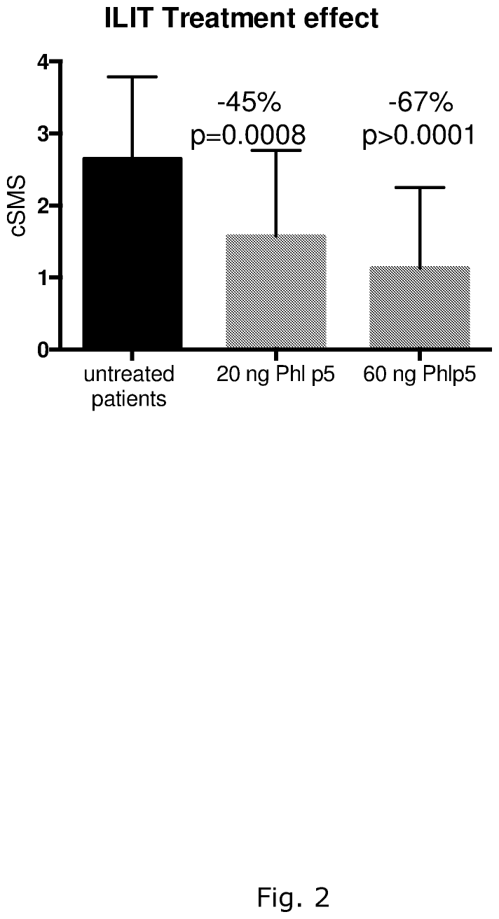

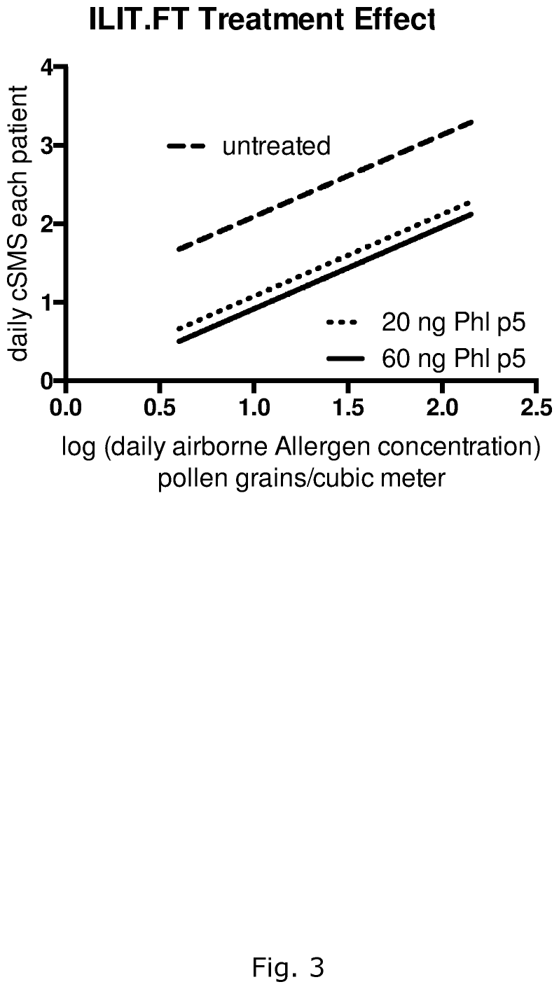

In an embodiment, a dosage containing/comprising 0.02 μg Phl p 5 to 0.08 μg Phl p 5 is to be administered to the subject, such as 0.04 μg Phl p 5 to 0.08 μg Phl p 5 (see also example 2 and FIGS. 1-3).

In an embodiment, the grass pollen extract is in the form of an extract of Phleum pratense pollen, or a mixture of pollen extracts from grass species such as Dactylis glomerata, Festiuca, Lolium perenne, Phleum pratense and Festuca pratensis, Secale cereale, or Dactylis glomerata, Anthxanthum odoratum, Lolium perenne, Phleum pretense and Poa pratensis or any other mixes of grass extract, preferably an extract from Phleum pratense pollen. Thus, Phl p 5 may preferably be delivered in the form of a grass pollen extract, which may also contain other grass allergens (different from Phl p 5).

In another embodiment, the grass pollen is an Alutard composition, preferably Alutard 225. In the example section, clinical trial experiments with Alutard 225 are described.

In yet an embodiment, the allergen composition comprises Phl p 5.

In yet an embodiment, the pharmaceutical composition comprises 200-400 SQ-U, preferably 250-350 SQ-U of grass pollen (grass allergen), and more preferably around 300 SQ-U.

In an embodiment, the pharmaceutical composition is for use in allergy vaccination.

In an embodiment, the pharmaceutical composition comprises aluminium hydroxide as an adjuvant, preferably the aluminium hydroxide being adsorbed to the grass allergen(s).

In a related embodiment, the pharmaceutical composition for use comprises 0.3 to 0.8 mg Al, more preferred 0.5-0.6 mg Al.

In another related embodiment, the pharmaceutical composition for use comprises 0.9 to 1.3 mg Al, more preferred 1-1.2 mg Al.

In a further embodiment, the pharmaceutical composition for use comprises a ratio of grass pollen (SQ-U) to Al (ion) (mg) in the range 200-400 SQ-U to 0.3 to 0.8 mg Al (ion), such as 200-400 SQ-U to 0.5 to 0.6 mg Al (ion), or such as 300 SQ-U to 0.5 to 0.6 mg Al (ion).

In yet a further embodiment, the pharmaceutical composition for use comprises a ratio of grass pollen (SQ-U) to Al (ion) (mg) in the range 200-400 SQ-U to 0.9 to 1.3 mg Al (ion), such as 200-400 SQ-U to 1.1 to 1.2 mg Al (ion), or such as 300 SQ-U to 1.1 to 1.2 mg Al (ion).

In an embodiment, Al is provided in the form of aluminium hydroxide.

In another embodiment, the adjuvant is selected from the group consisting of oxygen containing metal salts (aluminium hydroxide and calcium phosphate), heat-labile enterotoxin (LT), cholera toxin (CT), cholera toxin B subunit (CTB), polymerized liposomes, mutant toxins, e.g. LTK63 and LTR72, microcapsules, interleukins (e.g. IL-1[3, IL-2, IL-7, IL-12, INFy), GM-CSF, MDF derivatives, CpG oligonucleotides, LPS, MPL, phosphophazenes, Adju-Phos®, glucan, antigen formulation, liposomes, DDE, DHEA, DMPC, DMPG, DOC/Alum Complex, Freund's incomplete adjuvant, ISCOMs®, LT Oral Adjuvant, muramyl dipeptide, monophosphoryl lipid A (MPL), muramyl tripeptide, microcrystalline tyrosine and phospatidylethanolamine.

In yet an embodiment the pharmaceutical composition does not contain an adjuvant.

In an embodiment, the composition is administered as 2-4 dosages of 200-400 SQU, preferably, 250-350 SQ-U, and more preferably around 300 SQ-U, such as over a period of 6-10 weeks, such as three dosages ca. 4 weeks apart. Preferably the composition is administered as 2-4 dosages comprising 0.02-0.08 μg Phl p 5, such as 0.04-0.08 μg Phl p 5, preferably, 0.05-0.07 μg Phl p 5, and more preferably around 0.06 μg Phl p 5, such as over a period of 6-10 weeks, such as three dosages ca. 4 weeks apart. Again, more preferably Phl p 5 is in the form of an extract, preferably a grass pollen extract.

In an embodiment, the composition is administered as a dosage of a volume in the range 50-150 μl, such as 80-120 μl, or around 100 μl. 100 μl is a standard dose when administering directly into a lymph node.

In an embodiment, the composition is administered as 3 dosages over 8 weeks, such as three dosages ca. 4 weeks (two to eight weeks) apart.

In yet an embodiment Alutard is preferably Alutard 225.

In yet a further embodiment, the composition is administered into a lymph node.

In yet a further embodiment, the composition is administered into an inguinal lymph node.

In another embodiment, the subject is a mammal, such as livestock or pet, preferably the subject is a human.

In yet another embodiment, the subject suffers from grass pollen induced allergic disease. In a related embodiment, the subject suffers from allergic rhinitis, allergic conjunctivitis, allergic asthma, or allergic dermatitis caused by grass pollen allergen. Preferably the subject suffers from allergic rhinitis and/or allergic conjunctivitis.

In an embodiment, the pharmaceutical composition for use further comprises adjuvants and other excipients such as selected from the group consisting of solvents, emulsifiers, wetting agents, plasticizers, coloring substances, fillers, preservatives, viscosity adjusting agents, buffering agents, and the like.

In yet an embodiment the pharmaceutical composition for use is administered as a dosage of a volume in the range 50-150 μl, such as 80-120 μl, or around 100 μl. Thus, it is to be understood that the dosage of a volume in the range 50-150 μl, will then comprise the 100-400 SQ-U grass allergen, such as grass pollen extract, and optionally an adjuvant.

In another aspect, the invention relates to a method for the treatment or alleviation of grass allergy in a subject in need thereof, the method comprises administering to the subject a pharmaceutical composition comprising 100-400 SQ-U grass allergen, wherein the pharmaceutical composition is administered by Intralymphatic immunotherapy (ILIT) to a subject.

All patent and non-patent references cited in the present application, are hereby incorporated by reference in their entirety.

The invention will now be described in further details in the following non-limiting examples.

### EXAMPLES

### Example 1—Protocol

Aim of Study

To prepare a protocol for clinical trials.

Allergen is titrated from 1000 SQ-U that expert opinion suggests to one half log and one log less, and it is expected that the lesser amount of one half log less is going to be sufficient for directing a protective immune response, whilst leaving allergic responses to treatment at a minimum.

At the same time, the ratio of aluminium hydroxide to allergen is kept constant, as that appears to stabilise allergen in the lymph node. In experiments in which aqueous allergen was used (Lee S P et al. A Pilot Study of Intralymphatic Immunotherapy for House Dust Mite, Cat, and Dog Allergies. Allergy Asthma Immunol Res 2017; 9:272-277) allergic side effects were particularly prevalent. This may be due to the lack of experience indicated by “pilot” in the title, but may also be due to the lack of AIOH.

Materials and Methods

Allergen tested: Alutard 225 (ALK-Abello) (10,000 SQ-U)

Diluent: Supplied by (ALK-Abello)

For a 1000 SQ-U treatment, 200 μl of the study drug is drawn into a syringe. 100 μl is injected into the patient's inguinal lymph node.

For a 300 SQ-U treatment, 300 μl of the study drug is drawn into a syringe, and 700 μl diluent is drawn into the same syringe. The liquids are mixed by 3-fold inversion, and 800 μl of the liquid is expelled. 100 μl is injected into the patient's inguinal lymph node.

The dilution steps may be repeated to achieve an optimal lower dilution.

The injection of the allergen extract is performed aseptically by a member of staff from the Allergy center. The procedure is controlled and documented (filmed) by means of ultrasonography. Emergency utensils are provided.

### Example 2—Results

As also outlined above, it has been realized by the inventing team that a lower dosage of the allergen will have beneficial effect when used in ILIT treatment. Thus, a dosage of around 100-400 SQ-U (containing 0.02-0.08 μg Phl p 5), preferably such as 200-400 SQ-U, or 250-350 SQ-U and more preferably around 300 SQ-U (containing 0.06 μg Phl p 5) is considered optimal, e.g. by administering 3 times over 8 weeks.

Symptoms were registered on the cSMS scale defined by EAACI, (Pfaar 0 et al. Recommendations for the standardization of clinical outcomes used in allergen immunotherapy trials for allergic rhinoconjunctivitis: an EAACI Position Paper. Allergy 2014; 69:854-867).

The grass pollen season is defined as detailed by EAACI (Karatzas K et al. New European Academy of Allergy and Clinical Immunology definition on pollen season mirrors symptom load for grass and birch pollen-induced allergic rhinitis. Allergy 2018; 73:1851-1859).

To summarize, intralymphatic immunotherapy (ILIT) is a promising novel treatment of allergy (Senti et al.). Most treatment has been done with 200 ng Phl p5 (1 000 SQU Alutard) and attempts at increasing the dose to 2000 ng Phl p5 (10 000 SQU Alutard) did not succeed. Treatment with 600 ng Phl p5 (3 000 SQU Alutard) did not reduce symptoms appreciably (Hellkvist L. Intralymphatic immunotherapy in allergic rhinitis—Evaluating safety, efficacy and mechanisms. 2020). Therefore, treatment with a lower dose is considered more effective.

Materials and Methods

40 patients with grass pollen allergic rhinitis were recruited who were randomised to receive intralymphatic treatment with either 20 ng Phl p5 or 60 ng Phl p5. They were compared to a background of 312 patients who were not treated before or in this season.

See also example 1.

Results

The baseline patients had a mean cSMS of 2.649, and patients treated with 20 ng Phl p 5 had a reduction by 45% in cSMS to 1.417, patients treated with 60 ng Phl p 5 had an impressive reduction by 67% in cSMS to 1.129.

In the DBPCRT in 2016 (Skaarup S H et al. Intralymphatic immunotherapy improves grass pollen allergic rhinoconjunctivitis: A 3-year randomized placebo-controlled trial. J Allergy Clin Immunol 2021; 147:1011-1019.), the placebo group had a cSMS score of 4.5 (CI 3-6), the group treated with 3 injections of 200 ng Phl p5 had a cSMS score of 2.3 (CI 1.5-3), with a reduction of 48.8% and 11% side effects. In our comparable follow-up study in 2017 with 156 patients, we had a reduction in cSMS of 39.2% and 27% side effects (Skaarup S H et al. The number of successful injections associates with improved clinical effect in intralymphatic immunotherapy. Allergy 2021; 76:1859-1861).

In the de novo 3000 SQU trial in Stockholm (manuscript in Laila Hellkvists PhD, June 2020), the group on active treatment (600 ng Phl p5, n=16) had a reduction after treatment of 23.8% from 90.4 to 68.9. (this group sums the cSMS for each patient). The group on placebo had a reduction of 17.8% from 104.2 to 74.8. After treatment, the active group had 7.9% fewer cSMS than the placebo group. Thus, the most optimistic interpretation of treatment with 600 ng Phl p5 was a reduction of cSMS by 24%.

These accumulated results from the different studies are shown in FIGS. 1-3.

From this, it is concluded that around 20-60 ng major allergen Phl p 5 is the most effective dose for ILIT. The tested dosage of 60 ng provided the best results.

### CONCLUSION

The effect of such treatment (compared to a 1000 SQ-U dosage or higher):
Improved vaccination effect (longer and/or stronger); and/or
Lower side effects, such as lower allergic responses (due to a reduced injected dosages); and/or
Better clinical efficacy.

An even further improvement is considered to be present when the amount of adjuvant, is increased in the composition. As outlined above, the pharmaceutical composition may comprise a ratio of grass pollen (SQ-U) to Al (ion) (mg) in the range 200-400 SQ-U (containing 0.04-0.08 μg Phl p 5) to 0.3 to 0.8 mg Al (ion), such as 200-400 SQ-U to 0.5 to 0.6 mg Al (ion), or such as 300 SQ-U (containing around 0.06 μg Phl p 5) to 0.5 to 0.6 mg Al (ion).

1.-15. (canceled)

16. A method for treating or alleviating grass allergy in a subject, the method comprising administering to said subject a composition comprising 0.02-0.08 μg Phl p5, and an adjuvant, wherein the composition is administered by Intralymphatic immunotherapy (ILIT) to the subject.

17. The method according to claim 16, wherein Phl p5 is a grass pollen extract, wherein the grass pollen extract is in the form of an extract of Phleum pratense pollen or a mixture of pollen extracts from grass species.

18. The method according to claim 16, wherein the composition comprises 0.04-0.06 μg Phl p5.

19. The method according to claim 16, wherein the method is an allergy vaccination.

20. The method according to claim 16, wherein the composition comprises aluminium hydroxide as the adjuvant.

21. The method according to claim 16 wherein the composition comprises 0.3 to 0.8 mg Al.

22. The method according to claim 16, wherein the composition comprises 0.9 to 1.3 mg Al.

23. The method according to claim 16, wherein the composition comprises a ratio of grass pollen to Al (ion) (mg) comprising in the range 0.04-0.08 μg Phl p5 to 0.3 to 0.8 mg Al (ion).

24. The method according to claim 16, wherein the composition comprises a ratio of grass pollen to Al (ion) (mg) in the range 0.04-0.08 μg Phl p5 to 0.9 to 1.3 mg Al (ion).

25. The method according to claim 16, wherein the composition comprises 0.3 to 0.8 mg Al, and wherein Al is provided in the form of aluminium hydroxide.

26. The method according to claim 16, wherein the composition is administered into an inguinal lymph node.

27. The method according to claim 16, wherein the subject suffers from grass pollen allergy.

28. The method according to claim 16, wherein the subject suffers from allergic rhinitis, allergic conjunctivitis, allergic asthma, or allergic dermatitis caused by grass pollen allergen.

29. The method according to claim 16, wherein the composition further comprises additional adjuvants and other excipients.

30. The method according to claim 16, wherein the composition is administered at a dosage of a volume in the range 50-150 μl.

31. The method according to claim 16, wherein the composition is administered as 2-4 dosages comprising 0.02-0.08 μg Phl p 5 over a period of 6-10 weeks.

32. The method according to claim 16, wherein the composition comprises 0.05-0.07 μg Phl p 5.

33. The method according to claim 16, wherein the composition further comprises additional adjuvants and other excipients selected from the group consisting of solvents, emulsifiers, wetting agents, plasticizers, coloring substances, fillers, preservatives, viscosity adjusting agents, and buffering agents.

34. The method according to claim 16, wherein the subject is a human.
